(19) United States
(12) Patent Application Publication
(10) Pub. No.: US 2009/0107501 A1
(43) Pub. Date: Apr. 30, 2009

(54) SYSTEM AND METHOD OF MONITORING RESPIRATORY AIRFLOW AND OXYGEN CONCENTRATION

(76) Inventor: Ana Krieger, Rye, NY (US)

Correspondence Address: FAY KAPLUN & MARCIN, LLP, 150 BROADWAY, SUITE 702, NEW YORK, NY, 10038, US

(21) Appl. No.: 12/256,076

(22) Filed: Oct. 22, 2008

Related U.S. Application Data
(60) Provisional application No. 60/982,330, filed on Oct. 24, 2007.

(51) Int. Cl. A61M 16/10; A61B 5/145

(52) U.S. Cl. 128/204.23; 600/309

(57) ABSTRACT

Described is system and method of monitoring respiratory airflow and oxygen concentration. The system may include a first sensor producing data corresponding to an airflow in a respiratory system of a body; a second sensor producing data corresponding to an oxygen concentration in the body; a generator supplying a pressurized airflow; an oxygen source supplying oxygen; a conduit through which the pressurized airflow and oxygen are delivered to the respiratory system; and a processing arrangement for processing the data from the first and second sensors and for controlling the generator and the oxygen source based on the processed data.

### PRIORITY CLAIMS

This application claims the priority to the U.S. Provisional Application Ser. No. 60/982,330, entitled “System and Method of Monitoring Respiratory Airflow and Oxygen Concentration” filed on Oct. 24, 2007. The specification of the above-identified application is incorporated herewith by reference.

### BACKGROUND INFORMATION

Obstructive sleep apnea/hypopnea syndrome (OSAHS) is a well recognized disorder which may affect as much as 25-30% of the adult population. OSAHS is one of the most common causes of excessive daytime somnolence. OSAHS is most frequent in obese males, and it is the single most frequent reason for referral to sleep disorder clinics.

OSAHS is associated with all conditions in which there is anatomic or functional narrowing of the patient's upper airway, and is characterized by an intermittent obstruction of the upper airway occurring during sleep. The obstruction results in a spectrum of respiratory disturbances ranging from the total absence of airflow (apnea) to significant obstruction with or without reduced airflow (hypopnea, episodes of elevated upper airway resistance and snoring), despite continued respiratory efforts. The morbidity of the syndrome arises from hypoxemia, hypercapnia, bradycardia and sleep disruption associated with the respiratory obstruction event and arousals from sleep.

Positive Airway Pressure (PAP) therapy has been used to care for Obstructive Sleep Disordered Breathing (OSDB), which includes OSAHS, snoring, exaggerations of sleep-induced rises in collapsibility of the upper airway, and all conditions in which inappropriate collapsing of a segment of the upper airway causes significant un-physiologic obstruction to airflow. Such obstructions reduce oxygen in the blood and cause arousal from sleep. The availability of this non-invasive form of therapy has resulted in extensive publicity for sleep apnea/hypopnea and the appearance of large numbers of patients who previously may have avoided the medical establishment because of the fear of tracheostomy.

PAP therapy is directed to maintaining pressure in the collapsible portion of the airway at or above a critical tissue pressure. The critical tissue pressure is determined by a physician after diagnosing a patient with a respiratory sleep disorder. Such diagnoses are generally made after a polysomnography (PSG), an overnight evaluation at a sleep clinic. A PSG measures and records the changes in various physiological parameters that occur during sleep, such as oxygen concentration, chest wall and abdominal movement, respiratory air flow, heart rhythm using electrocardiography (ECG), electrical activity of the brain using electroencephalography (EEG), muscle activity using electromyography (EMG), and eye movement. After assessing these measurements, the physician will prescribe a titrated pressure for the PAP device, usually between 4 and 18 cm H₂0 or higher.

PAP devices treat respiratory conditions, such as sleep apnea, by delivering a stream of compressed air via a hose to a nasal pillow, nose mask or full-face mask, splinting the airway and keeping it open under pressure so that unobstructed breathing becomes possible. The Continuous Positive Air Pressure (CPAP) device is the conventional form of therapy, which remains at the prescribed pressure through the course of the night. However, a patient's physiological condition may vary from day to day or even throughout the night. The prescribed pressure of air flow may not be the ideal pressure every night or through the course of the evening.

A more recent technology, also used to treat Central Sleep Apnea and Cheyne-Stokes Respiration, called Assisted Servo Ventilation (ASV) may be utilized to improve respiratory disturbance present during sleep. Similarly, a bi-level positive airway pressure (BiPAP) device may be another treatment option for those patients who are suffering from more advanced sleep apnea and/or cases of nocturnal hypoventilation, where despite the absence of airway obstruction, ventilation is not sufficient to properly meet the demand and therefore needs to be enhanced. It may also be an option for patients who are non-compliant with CPAP therapy. For example, patients suffering from neuromuscular disease require ventilatory assistance and might not be able to breathe out against the CPAP pressure. The BiPAP allows more air to be breathed in and out by offering dual pressures, a higher pressure during inhalation and a lower pressure during exhalation.

As a patient may experience changes in air flow and oxygen concentration throughout the night, it would be beneficial for the CPAP and BiPAP device to adjust accordingly, as a prescribed pressure may be too high or too low at certain points of the evening. The monitoring of oxygenation levels during treatment with CPAP or BiPAP devices is used to determine the effectiveness of treatment. There are currently no PAP devices that allow for oxygenation monitoring or concomitant monitoring of respiration and oxygenation in a monitoring and treatment setting for either short term or long term use.

In cases of pediatric sleep apnea or respiratory disturbances in children, the additional monitoring of end-tidal carbon dioxide (CO₂) levels may be desirable, as it is considered a more sensitive technique for the monitoring and management of respiratory changes during wakefulness or sleep in a younger population. This technique is also important in the monitoring of other respiratory disturbances, such as hypoventilation or respiratory insufficiency in both the adult and pediatric groups, as it would assist in the management of the underlying condition.

### SUMMARY OF THE INVENTION

The present invention relates to a system and method of monitoring respiratory airflow and oxygen concentration. The system may include a first sensor producing data corresponding to an airflow in a respiratory system of a body; a second sensor producing data corresponding to an oxygen concentration in the body; a generator supplying a pressurized airflow; an oxygen source supplying oxygen; a conduit through which the pressurized airflow and oxygen are delivered to the respiratory system; and a processing arrangement for processing the data from the first and second sensors and for controlling the generator and the oxygen source based on the processed data.

In a further embodiment, the system may also include a third sensor producing data corresponding to an end-tidal CO₂ level in the body, the processing arrangement processing the data from the third sensor.

The method may include the following steps: measuring an airflow through a respiratory system of a body; producing airflow data based on the airflow measurement; measuring an oxygen concentration in the body; producing oxygen concentration data based on the oxygen concentration measurement; and determining an amount of pressurized airflow and oxygen to supply to the respiratory system based on the airflow data and the oxygen concentration data.

### BRIEF DESCRIPTION OF THE DRAWINGS

FIG. 1 shows an exemplary embodiment of a system according to the present invention, which monitors respiratory airflow, oxygenation and end-tidal CO₂ levels;

FIG. 2 shows an exemplary embodiment of a system according to the present invention, which monitors respiratory airflow, oxygenation and end-tidal CO₂ levels; and

FIG. 3 shows an exemplary embodiment of a method according to the present invention, which utilizes the system shown in FIGS. 1 and 2.

### DETAILED DESCRIPTION

The present invention may be further understood with reference to the following description and the appended drawings, wherein like elements are referred to with the same reference numerals. The present invention describes a system and method for monitoring respiratory airflow and oxygen concentration in positive air pressure (PAP) treatments, that include all forms of CPAP, Bi-level, ASV and face-mask ventilation techniques. The system and method may further monitor an end-tidal CO₂. These measures may be performed simultaneously. Although the present invention is described as a monitoring and/or treatment system and method for obstructive sleep apnea/hypopnea in pediatric and adult patients, it will be understood by those of skill in the art that the system and method of the present invention may be used for the monitoring of any respiratory disturbance during sleep, and during states of increased likelihood of airway collapsibility or ventilatory instability, including perioperative periods, central nervous system disease and under effect of extrinsic factors or medications affecting the respiratory drive and/or control, including airway dysfunction, sleep apnea, nocturnal hypoventilation, central apnea, Cheyne-Stokes respiration or in patients with chronic lung disease, emphysema, COPD or asthma.

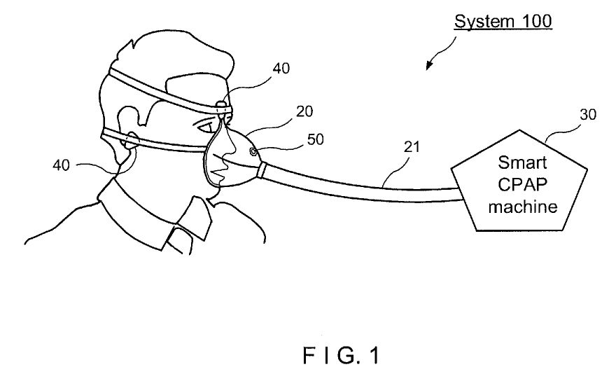

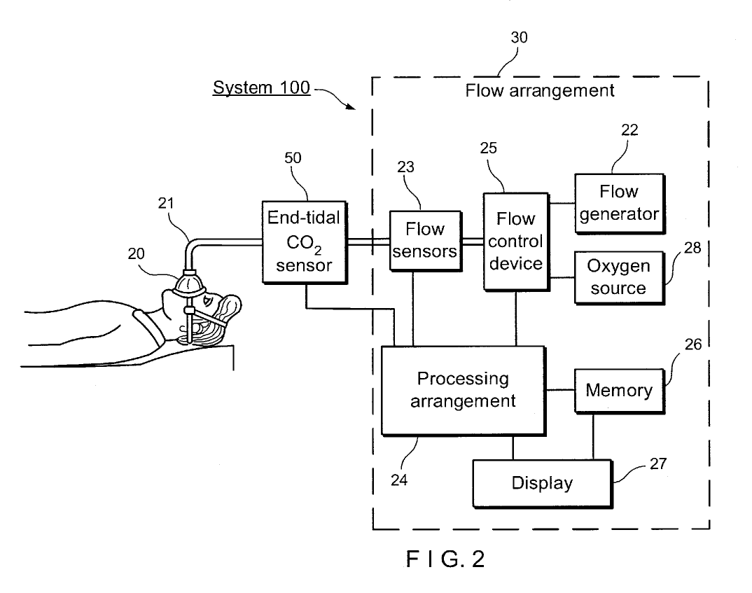

FIGS. 1 and 2 show an exemplary embodiment of a system 100 according to the present invention. The system 100 comprises a flow arrangement 30 that supplies pressurized airflow to a patient while monitoring the patient's respiratory airflow and oxygen levels via a flow sensor 23 and an oximetry sensor 40, respectively. In a further embodiment, the system 100 may also monitor an end-tidal CO₂ level and may thus further comprise an end-tidal CO₂ sensor 50. Pressurized airflow may be supplied to the patient via a mask 20, which is connected to a conduit such as a tube 21 to receive air from the flow arrangement 30. The mask 20 covers the patient's nose and/or mouth. FIG. 2 shows the flow arrangement 30 in greater detail. The flow arrangement 30 further comprises a flow generator 22 that provides airflow through the tube 21, an oxygen source 28 that provides oxygen through the tube 21, and a processing arrangement 24. The processing arrangement 24 outputs a signal to a conventional flow control device 25 to control a pressure and oxygen level applied to the flow tube 21.

Constructed in a conventional manner, the flow sensor 23 detects the rate of airflow to and from the patient. In an exemplary embodiment, the flow sensor 23 may be coupled to the tube 21, as shown in FIG. 2. In another embodiment, the flow sensor 23 may be placed in the mask 20. Alternatively, the flow sensor 23 may be positioned in a cannula placed near or inside the patient's nostril. In a further embodiment of the present invention, the flow sensor 23 may be internal or external to the generator 22 such that they are also able to detect the pressure supplied to the patient by the generator 22. It will be understood by those of skill in the art that the flow sensor 23 may be placed in a variety of areas as long as they are able to detect the rate of airflow and/or pressure to and from the patient. Signals corresponding to the airflow and/or pressure are provided to a processing arrangement 24 for processing.

The system 100 also includes an oximetry sensor 40, as shown in FIG. 1, that measures the patient's oxygen concentration. One technique by which sensor 40 monitors oxygen concentration is pulse oximetry. Pulse oximetry is a non-invasive method measuring the oxygenation of a patient's blood by placing a sensor 40 on a thin part of the patient's anatomy, such as the forehead or earlobe, in close connection with the flow arrangement 30. A light containing both red and infrared wavelengths is passed from one side to the other. Changing absorbance of each of the two wavelengths is measured, allowing determination of absorbances due to the pulsing arterial blood. The sensor 40 transmits oxygenation information to the processing arrangement 24 via cables or wirelessly, and this transmission may be done simultaneously or serially with the flow/pressure data provided by sensors 23. The data from sensor 40 may be transmitted back to the arrangement 30 by wireless transmission or wires extending through the mask 20 and tube 21. In the case of a wireless system, the oximeter may be located in other parts of the body, including fingers, toes, earlobes, or the forehead. Based upon the ratio of changing absorbance of the red and infrared light caused by the difference in color in oxygen-bound and oxygen-unbound blood hemoglobin, a measure of oxygenation (the percent of hemoglobin molecules bound with oxygen molecules) can be made by the processing arrangement 24.

In another embodiment, the system 100 may further comprise the end-tidal CO₂ sensor 50 to detect expiratory levels of carbon dioxide. The end-tidal CO₂ sensor 50 may be positioned within or adjacent to the mask 20. Alternatively, the end-tidal CO₂ sensor may be attached to a nasal cannula monitoring airflow and respiration. Information acquired by the end-tidal CO₂ sensor 50 is transmitted to the processing arrangement 24 of the flow arrangement 30 either simultaneously or serially with the flow/pressure data and the oxygen data provided by the sensors 23, 40.

Processing arrangement 24 may be loaded with software suitable for accepting data from the flow sensor 23, the oximetry sensor 40 and the end-tidal CO₂ sensor 50 and for performing analysis of one or more of the retrieved data. Based upon this analyzed information, the processing arrangement 24 may output a signal to a flow control device 25 to control the amount of oxygen being supplied to the patient through the tube 21 from the oxygen source 28 and/or the level of pressure at which the air is being supplied to the patient through the tube 21 from the flow generator 22. Those skilled in the art will understand that, for certain types of flow generator 22, the processing arrangement 24 may directly control the flow generator 22, the processing arrangement 24 may directly control the flow generator 22, instead of controlling airflow therefrom by manipulating the separate flow control device 25.

In a further embodiment of the present invention, flow arrangement 30 of system 100 may also include a memory 26 for storing and saving data received from the oximeter sensor 40, the flow sensor 23 and the end-tidal CO₂ sensor 50, as well as analyses of the data conducted by processing arrangement 24. The information may be further compiled into a report. Once the data and reports are stored in the memory 26, they may be downloaded by a physician or other healthcare provider at any time in order to assess the efficiency and effectiveness of the treatment as well as the need for possible adjustment. The downloaded report may be printed or displayed via an output means 27 such as a printer or a display. In another embodiment, results of the report may trigger an alarm or other response such that the physician or other healthcare professional may be notified of the results (e.g., in the case of abnormal results). An adjustment of the pressure may be automated or manually performed by the physician or healthcare provider based on the information supplied by the system 100 and an assessment of the report.

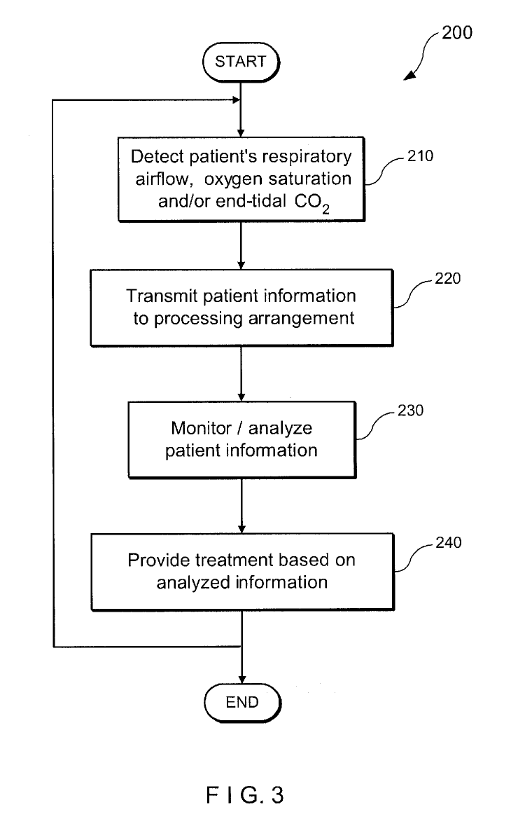

FIG. 3 shows an exemplary embodiment of a method 200 of the present invention. The system 100 is initiated by the placement of the mask 20 or a nasal cannula monitor the patient's nose and/or mouth and powering on of the generator 22, flow control device 25 and the processing arrangement 24. In the case of a mask placement with a pressurized device, as the PAP device continues to provide pressurized airflow to the patient, the patient's respiratory airflow/pattern is detected by pressure sensor 23 along with the patient's oxygen concentration by oximeter sensor 40 and the patient's end-tidal CO2 level by the end-tidal CO2 sensor 50, in step 210.

In step 220, the data airflow data from at least one of the pressure sensor 23, the oxygen concentration data from the oximeter sensor 40 and the end-tidal CO2 data from the end-tidal CO₂ sensor 50 are transmitted to the processor 24, which monitors the patient's respiratory airflow, oxygen levels and end-tidal CO₂ levels. The airflow data, the oxygen data and the end-tidal CO₂ data, or some combination thereof, may be synchronized. This information may be transmitted either wirelessly, which is generally preferable if oximetry sensor 40 is placed on an area of the body that is not in close connection with the processing arrangement 24, or via any suitable wired connection. Waves may be transmitted from the flow sensor 23 and the oximetry sensor 40 to the processor 24 via wires traveling through the mask 20 and the tube 21.

Once the information is transmitted, the data is monitored by the processor 24, in step 230, in order to analyze the patient's data. The processing arrangement 24 may utilize pre-stored patient data along with current data provided by the flow sensor 23, the oximetry sensor 40 and the end-tidal CO₂ sensor 50 in order to monitor the patient's status and/or identify abnormalities in the patient's data. In an exemplary embodiment of the invention, abnormalities may be identified by setting predetermined threshold values. If at least one of the patient's respiratory airflow, oxygen concentration and end-tidal CO2 level falls over or below the indicated threshold values, the patient data may be considered abnormal. Abnormal patient data may trigger an alarm or other response from the system 100. In another exemplary embodiment, patient information will be monitored by creating reports of the patient parameter values over time in order to assess trends in the patient's respiratory, oxygen and end-tidal CO₂ status, or a combination thereof.

In step 240, treatment is provided to the patient based on the analysis of the transmitted information. In an exemplary embodiment, the processor may automatically provide the appropriate treatment by increasing or decreasing the pressure of the airflow and/or the level of oxygen being provided. The processing arrangement 24 sends its instructions to the flow control device 25, which regulates the amount of oxygen being provided to the patient and the pressure that it is being provided at. If abnormalities are detected in the patient's data, the system 100 may respond by increasing the amount of oxygen being delivered to the patient, increasing the pressure of pressure of delivery to the patient, or both. The system will allow for uncovering previously undertreated or misdiagnosed conditions, in situations in which the patient's respiration is partially improved, by the use of the PAP device, but is not effective to fully restore oxygen levels in the blood. Alternatively, the system 100 may also respond by decreasing the amount of pressure delivered, decreasing the oxygen delivery to the patient, or both. It will be understood by those of skill in the art that the treatment provided by system 100 may adjust to various combinations of increased/decreased pressure and oxygen.

In another embodiment of the present invention, the synchronized data is stored in a memory 26 of the arrangement 30, without adjusting the preset oxygen level or pressure of delivery. The information may then later be downloaded into reports consisting of continuous oximetry analysis, number of events previously defined as percent oxyhemoglobin decrease over a specified period of time, mean values of oxygen and/or end-tidal CO2 or other analyses. The report may be printed or displayed via output means 27. This information may then be assessed by a physician or other healthcare professional in order to determine the effectiveness of, need for treatment or the need to make adjustments in treatment. Adjustments to the pressure and/or amount of oxygen being supplied may then be made manually by the physician or other trained or designated professional with expertise in this area. For cases in which the system 100 is used for monitoring purposes, initiation of therapy with the addition of PAP may also result from the analysis of the data derived from this system. Such information may also be used to compile clinical and statistical data to be used in research studies assessing the relationship between oxygen and/or end-tidal CO₂ levels during sleep and other medical or psychiatric conditions, as well as the efficacy of PAP treatments.

In a further embodiment of the present invention, the response may comprise both an auto-response of the system 100 that increases/decreases the pressure of air or oxygen delivery rate as well as storing the report of the information in memory for future download. In this embodiment, the system 100 is able to assess how the changes in pressure of delivery or amount of the oxygen being delivered affects the patient's respiratory airflow and oxygen levels through the course of the monitored period. Such an embodiment also has widespread clinical and research use, including pediatric and adult patients in an inpatient, ambulatory or outpatient setting.

It will be apparent to those skilled in the art that various modifications and variations can be made in the present invention without departing from the spirit or scope of the invention. Thus, it is intended that the present invention cover all modifications and variations of this invention when it comes within the scope of the appended claims and their equivalents.

What is claimed is:

1. A system, comprising:
a first sensor producing data corresponding to an airflow in a respiratory system of a body;
a second sensor producing data corresponding to an oxygen concentration in the body;
a generator supplying a pressurized airflow;
an oxygen source supplying oxygen;
a conduit through which the pressurized airflow and oxygen are delivered to the respiratory system; and
a processing arrangement for processing the data from the first and second sensors and for controlling the generator and the oxygen source based on the processed data.

2. The system of claim 1, further comprising a third sensor producing data corresponding to an end-tidal CO₂ level in the body, the processing arrangement processing the data from the third sensor.

3. The system of claim 1, wherein the conduit connects one of (i) a mask covering at least one of a mouth and a nose of a body and (ii) a nasal cannula located at one of an opening of the mouth and the nose to the generator for one of monitoring and supplying pressurized airflow to the respiratory system.

4. The system of claim 1, wherein the processing arrangement responds to the processed data by one of (i) adjusting an amount of oxygen being supplied, (ii) adjusting one of an amount of airflow being supplied and a pressure of the airflow being supplied, (iii) generating an alarm and (iv) printing a report.

5. The system of claim 1, further comprising a memory to store at least one of a data received by the processing arrangement and the processed data.

6. The system of claim 5, wherein at least one of the processed data and the data stored in the memory is downloaded in a report.

7. The system of claim 6, wherein the report contains at least one of a continuous oximetry analysis and a number of events predefined as percent hemoglobin decrease over a specified period of time.

8. The system of claim 3, wherein the first sensor is placed in at least one of the mask, the conduit and the nasal cannula.

9. The system of claim 1, wherein the second sensor is placed on at least one of a finger, a toe, an earlobe, and a forehead of the body.

10. The system of claim 1, wherein the second sensor measures data corresponding to oxygen concentration using pulse oximetry.

11. The system of claim 1, wherein the processing arrangement receives the data from the first and second sensors via at least one of a wireless transmission and a wired transmission.

12. The system of claim 1, wherein the processing arrangement identifies respiratory abnormalities.

13. The system of claim 12, wherein respiratory abnormalities are identified by comparing the data from the first and second sensors with threshold data stored in the processing arrangement.

14. The system of claim 1, wherein the second sensor produces the data corresponding to oxygen concentration concomitantly with the data corresponding to airflow.

15. The system of claim 2, wherein the third sensor produces the data corresponding to the end-tidal CO₂ level concomitantly with the data corresponding to airflow and oximetry.

16. A system, comprising:
a first sensor producing data corresponding to an airflow in a respiratory system of a body;
a second sensor producing data corresponding to an oxygen concentration in the body; and
a processor retrieving data from the first and the second sensors and performing an analysis of the data.

17. The system of claim 16, wherein at least one of the airflow and the oxygen concentration is manually adjusted based on the analysis of the first sensor data and the second sensor data.

18. The system of claim 16, further comprising a third sensor producing data corresponding to an end-tidal CO₂ level, the processor retrieving data from the third sensor.

19. A method, comprising the steps of:
measuring an airflow through a respiratory system of a body;
producing airflow data based on the airflow measurement;
measuring an oxygen concentration in the body;
producing oxygen concentration data based on the oxygen concentration measurement; and
determining an amount of pressurized airflow and oxygen to supply to the respiratory system based on the airflow data and the oxygen concentration data.

20. The method of claim 19,further comprising:
measuring an end-tidal carbon dioxide level in the respiratory system and producing end-tidal carbon dioxide data based on the end-tidal CO₂ measurement.

21. The method of claim 19, further comprising the step of:
placing one of a mask over at least one a nose and a mouth of the body and a nasal cannula at an opening of one of the nose and the mouth, the mask and the nasal cannula connecting to a conduit through which the pressurized airflow and oxygen is delivered to the respiratory system.

22. The method of claim 19, wherein after the determining of the amount of pressurized airflow and oxygen, the method further comprises one of (i) adjusting one of a pressure of the airflow and an amount of the airflow being supplied and (ii) adjusting the amount of oxygen being supplied.

23. The method of claim 19, further comprising the step of:
storing the airflow data and the oxygen concentration data in a memory.

24. The method of claim 23, further comprising the step of:
downloading the stored airflow data and oxygen data in a report.

25. The method of claim 19, further comprising the step of:
placing a sensor to measure and produce the airflow data in at least one of the mask or the conduit.

26. The method of claim 19, further comprising the step of:
placing a sensor to measure and produce the oxygen concentration data on at least one of a forehead, an earlobe, a finger, and a toe of the body.

27. The method of claim 19, wherein the oxygen concentration is measured using pulse oximetry.

28. The method of claim 19, further comprising the step of:
transmitting the airflow data and the oxygen concentration data by at least one of a wireless transmission and a wired transmission.

29. The method of claim 19, further comprising the step of:
identifying respiratory abnormalities based on the oxygen concentration data and the airflow data, wherein the oxygen concentration data and the airflow data are compared to threshold values.
